(19) United States
(12) Patent Application Publication
(10) Pub. No.: US 2017/0182489 A1
(43) Pub. Date: Jun. 29, 2017

(54) RECEIVER CONTAINER AND RECEIVER UNIT FOR RECEIVING BODY FLUID

(71) Applicant: Greiner Bio-One GmbH, Kremsmuenster (AT)

(72) Inventors: Franz Ebetsberger, Kremsmuenster (AT); Maximilian Mizelli, Ried im Traunkreis (AT)

(73) Assignee: Greiner Bio-One GmbH, Kremsmuenster (AT)

(21) Appl. No.: 15/302,561

(22) PCT Filed: Apr. 8, 2015
(86) PCT No.: PCT/AT2015/050089

§ 371 Date: Jan. 20, 2017

(30) Foreign Application Priority Data
Apr. 9, 2014 (AT) A 50269/2014

(51) Int. Cl. B01L 3/00; A61B 5/15

(52) U.S. Cl. CPC B01L 3/5021; A61B 5/150351; B01L 2300/0832; B01L 2300/0858; B01L 2300/12

(57) ABSTRACT

The invention relates to a receiver container (2) for receiving body fluid, in particular blood, comprising an open end (4), an end (5) closed by a base wall (6) and a container wall (7) which extends therebetween and defines a longitudinal axis (8). The container wall (7) has an internal surface (9) and an external surface (11) spaced apart therefrom by a wall thickness (10). The receiver container (2) is made from a plastic material. At least one reinforcement rib (27) is arranged on at least one first subsection (28) of the internal surface (9) of the container wall (7) and protrudes towards the longitudinal axis (8), said first subsection (28) extending from the region of the closed end (5) and in the direction towards the open end (4). The invention also relates to a receiver unit (1) comprising such a receiver container (2) which is closed by a closure device (3).

The invention relates to a receiver container as well as a receiver unit comprising such a receiver container and a closure device closing its open end, which is used for receiving body fluid, in particular blood, as defined in claims 1 and 22.

When vessels for taking blood specimens were first produced, they were made from glass and also had a closure device for closing the open end. The receiving chamber was also evacuated to enable blood to be drawn in when taking a blood specimen.

WO 89/09735 A1 and EP 0 419 490 B1 based on it as well as U.S. Pat. No. 5,275,299 A describe a number of closure devices for cylindrical housings made from a plastic material which can be evacuated in particular. For reasons of stability and to obtain barrier properties, the thickness of the wall of the housing was ca. 1.0 mm and even larger. The standard dimensions of such tubes were based on an external diameter of 13 mm and the standard length was 75 mm or 100 mm. There were also some tubes with a standard diameter of 16 mm for a standard length of 100. In terms of standard dimensions, standard diameter or standard length should be understood as meaning dimensions corresponding to the external dimensions and due to the conicity that is usually provided, there is a slight reduction in the external diameter from the open end to the closed end. Not only did this enable sufficient heat resistance and barrier properties to be obtained, but also a straightness within the tolerance range and a lack of distortion of these more solid wall thicknesses of the container wall.

The underlying objective of this invention is to guarantee sufficient dimensional stability as well as high heat resistance for receiver containers made from a plastic material and having thin or very thin wall thicknesses used for directly receiving body fluid, in particular blood. Thin wall thickness should be understood as meaning dimensions of less than/equal to 0.8 mm.

This objective of the invention is achieved by the features defined in claim 1. The advantages of the features defined in claim 1 reside in the fact that the internal surface against which the body fluid, in particular blood, is in contact in the receiving chamber for storage purposes can be sufficiently reinforced by providing the at least one reinforcement rib. As a result, whilst keeping the same standard external dimensions in terms of diameter and longitudinal extension, the container wall can be additionally strengthened. As a further consequence of this, the wall thickness can be further reduced which means that a saving can be made on raw material resources on the one hand and the amount of material to be subsequently disposed of can be reduced on the other hand. Depending on the disposition and orientation of the reinforcement ribs, which might also be described as stiffening ribs, the strength, straightness and roundness as well as the ability to retain shape can be positively influenced. Furthermore, by providing the at least one reinforcement rib, the surface area of the internal surface can be made larger, which thus results in a larger reaction surface in the interior. Since the internal surface of the receiver container is usually coated with chemicals, the desired reaction can be speeded up due to the increased size of the surface. This being the case, the blood will coagulate more rapidly after the filling operation than has been the case in the past, for example. Furthermore, due to the increased volume of the receiving chamber, a larger quantity can be accommodated relative to the standard dimensions than in the past whilst retaining the same external dimensions. As a further consequence, however, the negative pressure in the receiving chamber in absolute terms can be selected so as to be lower than is the case with conventional container units of the same standard dimensions.

Also of advantage is an embodiment defined in claim 2 because a uniform strengthening or stiffening of the container wall can be obtained along the internal wall. Due to the spiral-shaped, in particular helical, longitudinal extension, a strengthening and stiffening effect can be achieved not only around the circumference but also across the longitudinal extension of the entire container wall.

Also of advantage is an embodiment defined in claim 3, because an even more uniform strengthening and stiffening effect can be achieved. This makes it possible to opt for extremely thin wall thicknesses and a high stiffening effect can additionally be achieved by providing a number of spiral-shaped reinforcement ribs.

With the embodiment defined in claim 4, the receiver container can be easily adapted to a range of different applications and conditions based on the choice of the number of reinforcement ribs disposed on the internal surface and the density of their disposition. The smaller the pitch value selected, the greater the proportion of surface area occupied by the reinforcement ribs on the internal surface of the container wall.

Based on another embodiment defined in claim 5, a circumferential, continuous stiffening effect is obtained at different levels on the container wall along the longitudinal axis. As a result, the wall thickness can also be reduced in the region of the container wall and the requisite strength properties can be achieved by providing a number of reinforcement ribs.

Also of advantage is another embodiment defined in claim 6 because the strengthening effect can easily be adapted to different application conditions depending on the size of the width of the reinforcement ribs.

The advantage of the embodiment defined in claim 7 is that depending on the degree of protrusion, the stiffening effect of the at least one reinforcement rib for the container wall is still sufficient even if the wall thickness is reduced. To make it easy to remove the mold core and thus pull out or remove the receiver container from the mold core, the height or extension of the protrusion is adapted to the angle of inclination of the internal wall as viewed in axial section relative to the longitudinal axis.

As a result of another embodiment defined in claim 8, the external dimensions or standard dimensions of receiver containers used in the past can still be used and only the wall thickness needs to be adapted.

Likewise based on the embodiment defined in claim 9, the standard dimensions can remain unchanged without the need to make modifications to the standard laboratory devices used.

Also of advantage is an embodiment defined in claim 10 because a receiver container that has sufficient strength properties can be obtained in spite of the relatively smaller thickness selected for the wall thickness in conjunction with the reinforcement rib or ribs on the inner face or internal surface. Accordingly, it is possible to get by using a lesser amount of material. In addition, however, the increased capacity means that whilst opting for a receiver container of the same standard size, a standard receiver container of the type used in the past can be used to accommodate larger quantities in the receiving chamber. In certain situations, therefore, there is no need to use a receiver container of the next largest standard size because a larger quantity can now also be received in the receiver container of the smaller standard size. This also means that the number of types used in the laboratory can be reduced.

Based on an embodiment defined in claim 11, a relatively thin container wall is obtained, on the internal face of which the reinforcement ribs needed to impart the strengthening effect are disposed. However, this also results in a uniform shrinkage and/or cooling behavior, which means even less susceptibility to warping and hence improved straightness of the container wall relative to the longitudinal axis.

Based on another advantageous embodiment defined in claim 12, an exactly circumferentially extending sealing surface is obtained in the region of the open end, thereby preventing the undesired formation of passages between the sealing surface of the sealing stopper and the sealing section.

In this respect, an embodiment defined in claim 13 has proved to be of advantage because an exactly formed sealing surface can be obtained on the internal surface of the container wall. However, due to the virtually or approximately cylindrically shaped sealing section, an exact radial supporting effect is obtained for the sealing stopper inserted in the open end. This more easily prevents the latter from being inadvertently pulled out of the receiving chamber.

Due to the embodiment defined in claim 14, a more rigid open end that is more resistant to deformation can be obtained, thereby enabling better sealing of the receiving chamber to be maintained when the sealing stopper is inserted, even after a longer period of time. Furthermore, this also prevents the smaller closed end of receiver containers that are not yet closed from penetrating the open end of another in a random orientation when being conveyed.

Based on another advantageous embodiment defined in claim 15, the diameter of the opening of tubes with the otherwise standard wall thickness can be kept the same, which means that standard closure stoppers can continue to be used requiring no modification. There is therefore no need to make changes to the closure stopper and the entire closure device can continue to be used without having to make changes. Furthermore, however, the closed end of receiver containers that are not yet closed can be prevented from penetrating the open end of another which could cause mutual jamming and the formation of a stack.

In this respect, an embodiment defined in claim 16 has proved to be of advantage because sufficient strength can be imparted to the base of the receiver container which is subjected to a high degree of stress during centrifuging in spite of the fact that the wall thickness has been reduced.

Based on the embodiment defined in claim 17, undesired deformation of the receiver container and any damage which might be caused as a result can be prevented during centrifugation during which the base or closed end is usually supported on the centrifuge.

Based on another advantageous embodiment defined in claim 18, the stress which occurs during centrifugation can be withstood without causing damage or deformation to the base region depending on the increased wall thickness, even without providing additional reinforcement ribs and/or ribs or webs.

Also of advantage is an embodiment defined in claim 19 because the receiver container can be easily adapted to a range of different application conditions based on an appropriate choice of plastic material.

As specified in claim 20, the material itself already contains at least one appropriate additive intended to impart sufficient barrier properties in spite of the reduced wall thickness. Depending on the choice and/or combination of barrier additives, not only can the gas barrier properties be influenced accordingly, the liquid barrier properties can also be influenced, in particular improved. The purpose of an increased gas barrier property is that it enables the vacuum or negative pressure which usually prevails in the receiving chamber to be maintained for a longer period of time or for a longer duration.

The advantage of the embodiment defined in claim 21 is that depending on the nature of the barrier coating selected, the barrier properties can be increased on the one hand and the purity of the specimen accommodated in the receiving chamber can be guaranteed on the other hand. This makes it possible to obtain even more exact specimen results because the material chosen for making the receiver container does not come into direct contact with the specimen and no undesired constituents or softeners or similar from it are able to get into the specimen.

However, the objective of the invention can also be achieved independently by the features defined in claim 22. The advantages obtained as a result of the combination of features defined in this claim reside in the fact that a unit specifically for receiving and taking specimens of blood in particular can be obtained.

Finally, another embodiment as defined in claim 23 is possible whereby a perfect inflow of the body fluid to be accommodated, in particular blood, into the receiving chamber can be obtained due to the otherwise low pressure differences right until the total filling quantity is reached. Furthermore, due to the increased volume of the receiving chamber, the reduced pressure can also be kept at a lower level than is the case with the receiver units used to date. Accordingly, the blood flows into the receiving chamber more slowly than is the case with receiver units used to date, as a result of which hemolysis, amongst other things, can be reduced or prevented altogether.

To provide a clearer understanding, the invention will be described in more detail below with reference to the appended drawings.

These are highly simplified schematic diagrams illustrating the following:

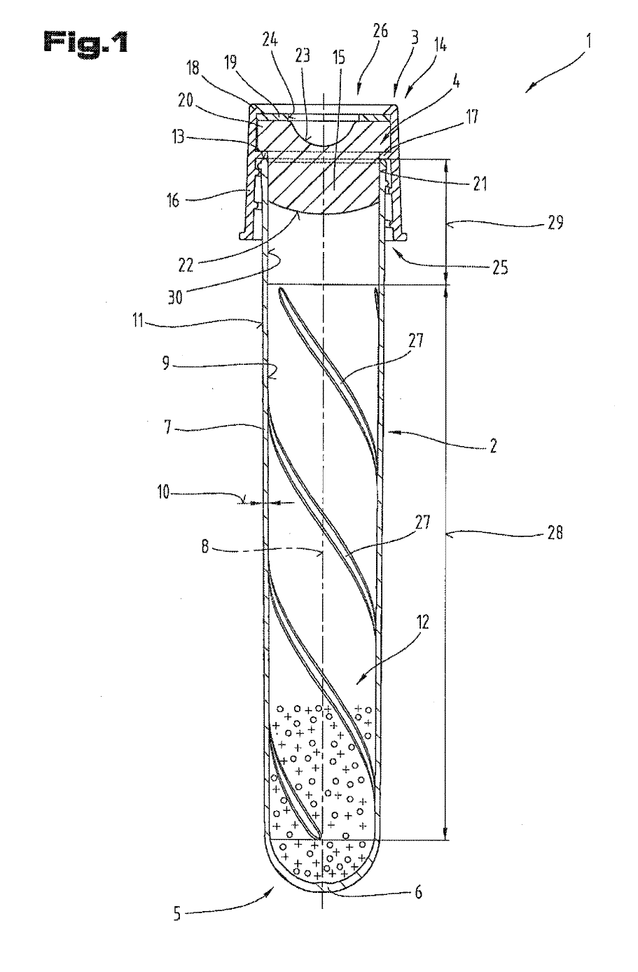

FIG. 1 a receiver unit with receiver container and closure device, viewed in axial section;

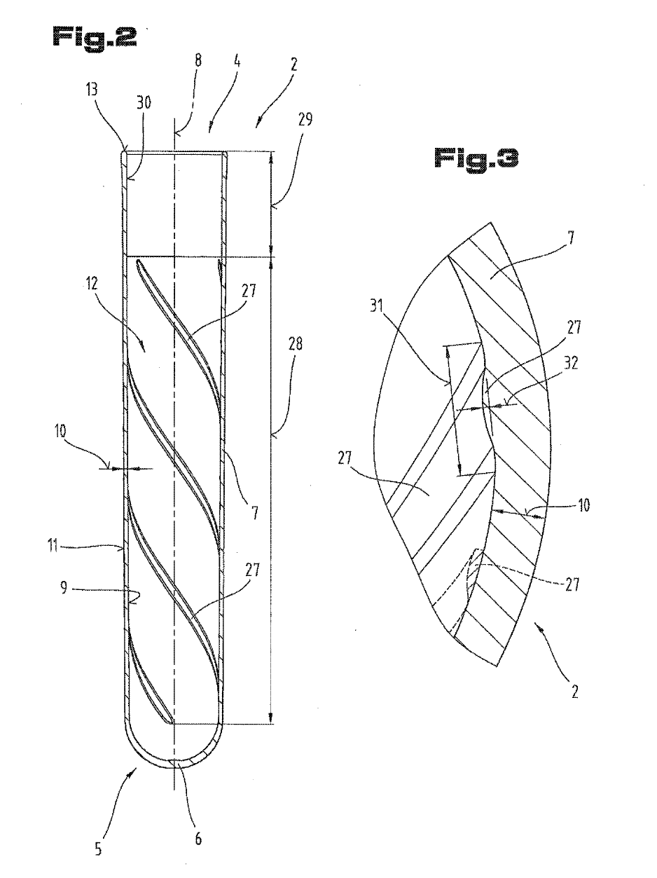

FIG. 2 the receiver container having spiral-shaped reinforcement ribs illustrated in FIG. 1, viewed in axial section;

FIG. 3 a reinforcement rib of the receiver container illustrated in FIGS. 1 and 2, viewed in cross-section and on an enlarged scale;

FIG. 4 another possible embodiment of a receiver container, viewed in axial section;

FIG. 5 a different receiver container having reinforcement ribs extending circumferentially around the internal circumference, viewed in axial section;

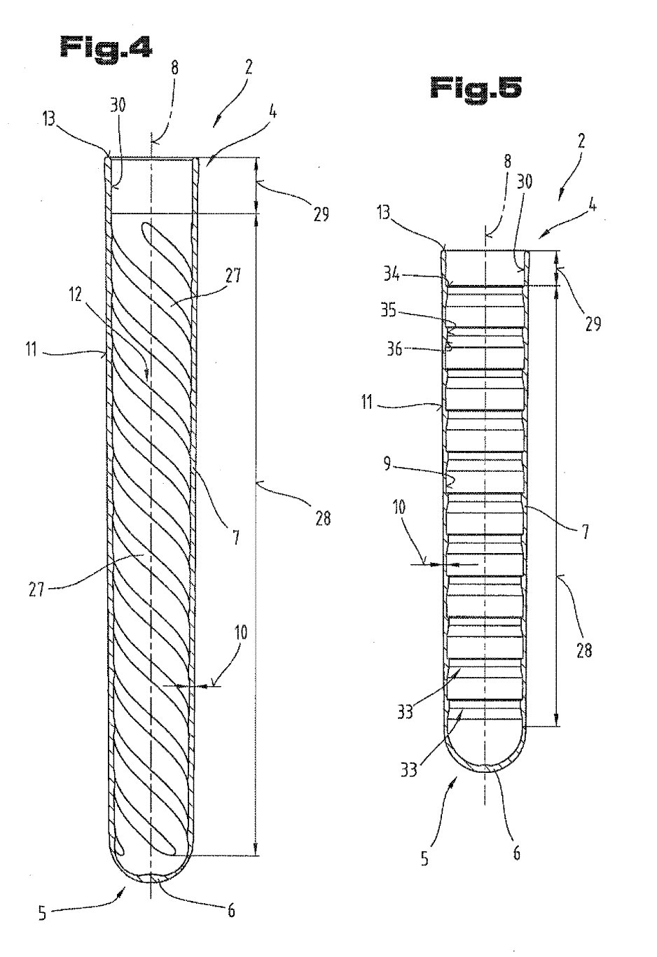

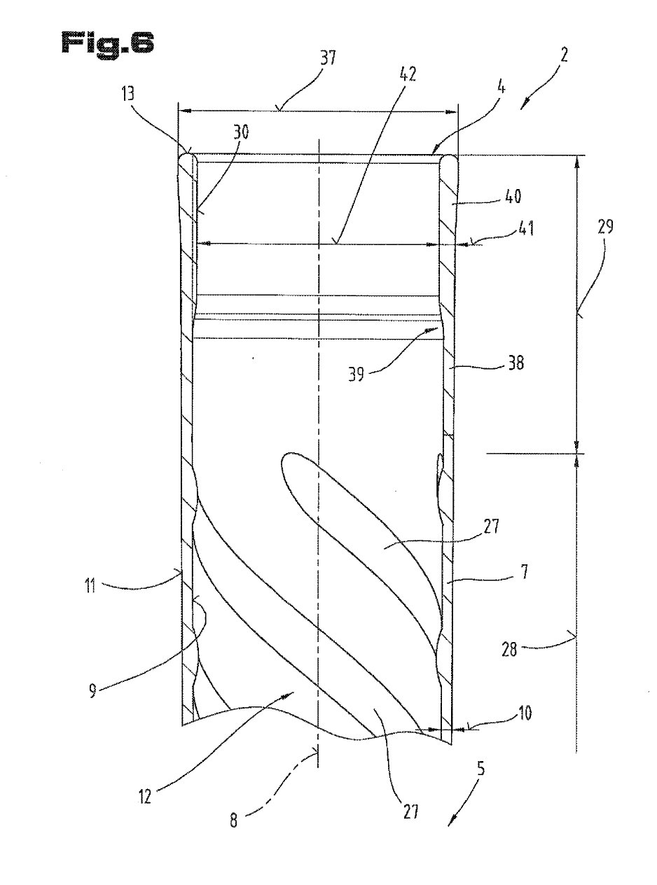

FIG. 6 a subsection of one possible embodiment of the receiver container in the region of its open end, viewed in axial section and on an enlarged scale;

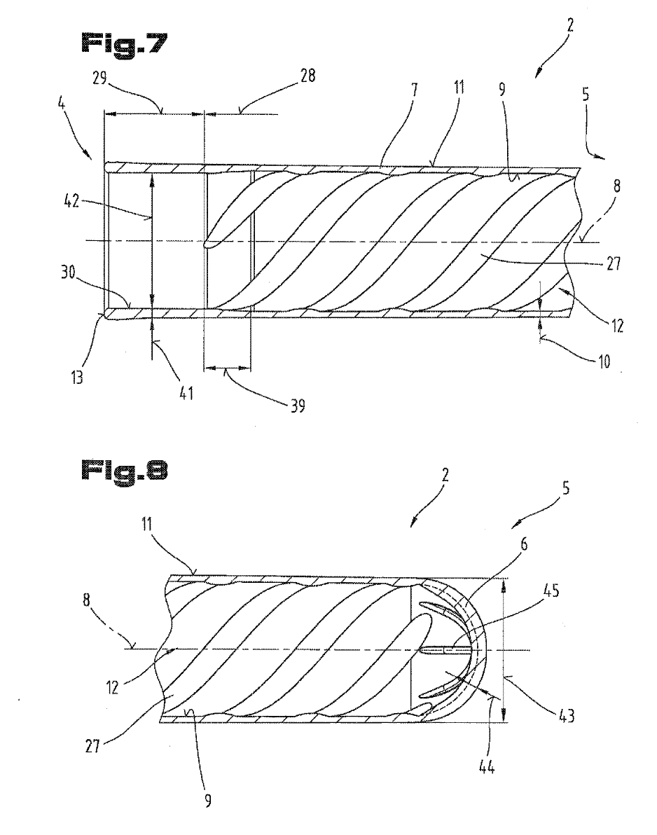

FIG. 7 a subsection of another possible embodiment of the receiver container in the region of its open end, viewed in axial section and on a larger scale;

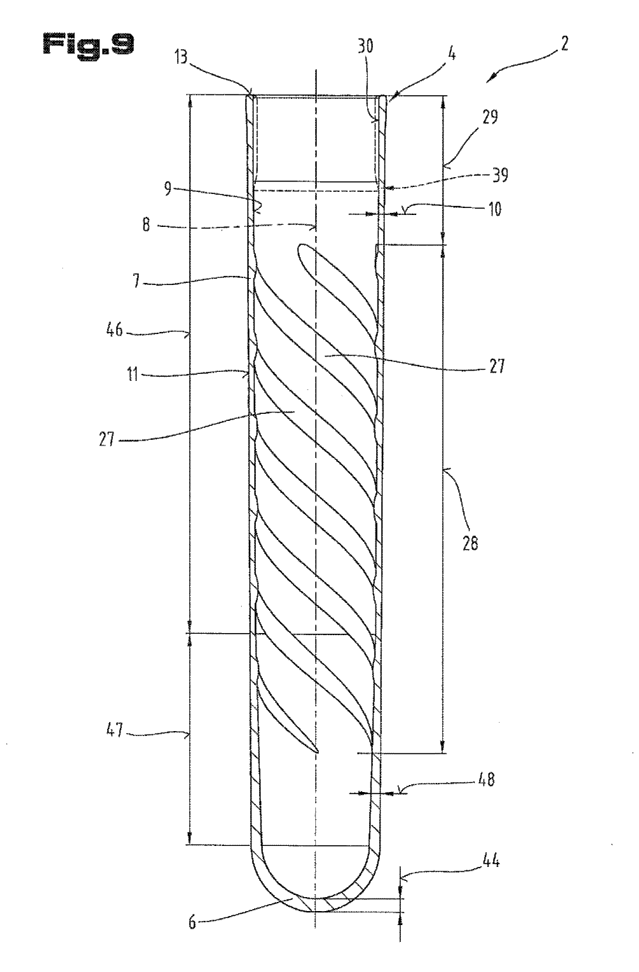

FIG. 8 a subsection of another possible embodiment of the receiver container in the region of its closed end, viewed in axial section and on a larger scale;

FIG. 9 another possible embodiment of a receiver container, viewed in axial section.

Firstly, it should be pointed out that the same parts described in the different embodiments are denoted by the same reference numbers and the same component names and the disclosures made throughout the description can be transposed in terms of meaning to same parts bearing the same reference numbers or same component names. Furthermore, the positions chosen for the purposes of the description, such as top, bottom, side, etc., relate to the drawing specifically being described and can be transposed in terms of meaning to a new position when another position is being described.

The expression “in particular” used hereafter should be understood as relating to a possible, more special embodiment or more exact specification of an object or method step, but this does not necessarily mean that it is necessarily a preferred embodiment or a preferred approach.

FIG. 1 illustrates a receiver unit 1, e.g. for a mixture of at least two different constituents or media, such as, for example, body fluids, or tissue parts or tissue cultures, although these will not be described or specified in further detail. The receiver unit 1 is preferably used for collecting and taking specimens of blood and may also be described as a holder device or blood specimen tube.

The receiver unit 1 comprises an approximately cylindrically shaped receiver container 2 and a closure device 3. This receiver container 2 together with the closure device 3 can also be used as an evacuated blood specimen tube and may be based on a range of different embodiments. The receiver unit 1 is preferably supplied to the user as an assembled unit, usually with the interior evacuated ready for use. However, it would also be possible for the receiver container 2 to be supplied to the user separately from the closure device 3 ready for filling.

The receiver container 2 has two mutually spaced apart ends 4, 5, and based on this example of an embodiment, the end 4 is open and the end 5 is closed by means of a base wall 6 in particular. Generally speaking, the base wall 6 may also be described simply as a base. The three-dimensional shape of the base wall 6 in this particular example of an embodiment is dome-shaped or cambered towards the end remote from the open end. However, the base wall 6 may also be formed by a spherically shaped terminating wall. Another option would be to provide the base wall 6 as a virtually flat end wall. Independently of these options, however, the base or base wall 6 need not necessarily be designed integrally with the receiver container 2 and instead the end 5 could also be provided with a separate closure device for closing the end, although this will not be described and illustrated here.

What in this instance is the open end 4 can be closed if necessary by means of the closure device 3 illustrated on a simplified basis here, which may be of the type described in EP 0 445 707 B1, EP 0 419 490 B1, U.S. Pat. No. 5,275,299 A, U.S. Pat. No. 5,495,958 A and U.S. Pat. No. 5,522,518 A, for example. To avoid unnecessary repetition, the disclosures relating to the design of the cap, the sealing device, the coupling device between the cap and sealing device and the cap and receiver container 2 as well as the provision of the possible retaining ring are incorporated in this application by way of reference.

The receiver container 2 further comprises a container wall 7 extending between the open end 4 and the closed end 5, thus defining a longitudinal axis 8, which container wall 7 has an internal surface 9 and an external surface 11 spaced apart therefrom by a wall thickness 10.

The container wall 7 and the base wall 6 bound a receiving chamber 12 in which the body fluid, in particular blood, is directly accommodated and sits in direct contact with the internal surface 9. To facilitate filling or even make it possible at all, the receiving chamber 12 sealed off from the external ambient atmosphere can be reduced to a pressure below atmospheric pressure.

The receiver container 2 may be bottle-shaped, vial-shaped, barrel-shaped or such like, for example, and may be made from a range of different plastic materials. The container wall 7 is of a predominantly tubular shape and has a slight conicity starting from the open end 4 in the direction towards the closed end 5. It is preferable to select a plastic which is fluid-tight, in particular waterproof, and optionally gas-tight. This may be selected, for example, from the group comprising polyethylene terephthalate (PET), polyethylene (PE), polypropylene (PP), polystyrene (PS), polycarbonate (PC), high-density polyethylene (HD-PE), acrylonitrile-butadiene-styrene copolymers (ABS), polyamide (PA) or a combination thereof. Another option, however, is to mix polyamide (PA) in a proportion by weight or a proportion by volume of ca. 1% to 10% with the polyethylene terephthalate (PET) material prior to the production process.

The inner surface, namely the internal surface 9, and the container wall 7 with the inner clearance dimension therefore determine an internal cross-section, which may be of various cross-sectional shapes, e.g. circular, elliptical, oval, polygonal, etc. The shape of the external cross-section may also be circular, elliptical, oval, polygonal, etc. but it is also possible for the shape of the external cross-section to be of a different shape from that of the internal cross-section. However, it is preferable if the cross-section is circular.

It is of advantage if the inner first dimension of the receiver container 2, starting from the open end 4 towards the other closed end 5 spaced apart therefrom has a minimal constant taper towards the inner other dimension. The reason for this is that if the receiver container 2 is manufactured by means of an injection casting process, for example, it can be easily removed from the injection casting mold. The taper or taper angle relative to the inner oppositely lying surfaces of the receiver container 2 is between 0.1° and 3.0°, preferably between 0.2° and 1.0°. At this stage, it should be pointed out that the described dimensions relate to the distance between the mutually opposite internal and external surfaces of the components, the diameter, the circumference along an envelope or enveloping line as well as the cross-section or cross-sectional surface respectively in one of the planes oriented perpendicular to the longitudinal axis 8 and the same spatial direction is always used to determine the dimensions.

As may also be seen from this diagram, the open end 4 has an open end face 13 which can be closed by the closure device 3 and opened again if necessary. To this end, the closure device 3 comprises a cap 14 enclosing the open end face 13 and a sealing device retained therein, for example a sealing stopper 15, made from a highly elastic and self-closing material which can be pierced, e.g. pharmaceutical rubber, silicone rubber or bromobutyl rubber. This cap 14 is usually disposed concentrically with respect to the longitudinal axis 8 and is formed by a tubular or annular cap casing 16. Coupling means may be provided between the cap 14 and sealing device, for example coupling parts of a coupling device. The cap 20 may have at least two projections 17, 18 disposed around certain regions of the internal circumference and optionally a retaining ring 19, and the sealing stopper 15 has a shoulder 20 projecting out from at least certain regions of its external circumference.

In this example of an embodiment, the sealing stopper 15 has a circumferentially extending, approximately cylindrical sealing surface 21 disposed approximately concentrically with respect to the longitudinal axis 8, which when inserted in the receiving chamber 12 in its sealing position in the region of the open end 4 sits in abutment with the internal surface 9 of the receiver container 2. In this portion, therefore, the internal surface 9 of the receiver container 2 is designed with a surface quality that is suitable for a sealing surface. The sealing stopper 15 also has another sealing surface 22 intended to be pierced which, in conjunction with the sealing surface lying against the internal surface 9 and sealing surface 21, closes and/or seals off the receiving chamber 12 of the receiver container 2 from the external environment at its open end face 13. Providing the projection 17 in at least certain regions between the shoulder 20 projecting out from the sealing surface of the sealing stopper 15 and the open end face 13 of the receiver container 2 prevents the shoulder 20 from sticking or strongly adhering to the end face 13.

It may also be preferable if the sealing stopper 15 has a recess 23 on the side facing the retaining ring 19 with a cross-sectional surface that is approximately the same as an opening 24, and in terms of its dimensions, this opening 24 is designed so that a cannula, not illustrated here, is able to pass through unhindered and then pierce the sealing device or sealing stopper 15.

Above all, the seal provided by the closure device 3 for the open end face 13 of the receiver unit 1 can be further improved if an external diameter of the sealing stopper 15 in the region of its sealing surface in the relaxed state outside of the receiver container 2 is bigger than the internal dimension of the receiver container 2 in the region facing the sealing stopper 15 of the sealing device.

It is also of advantage if the cap casing 16 is designed as a cylindrical casing or alternatively frustoconical casing to ensure that the cap casing 16 extends round the region of the top open end face 13.

In addition to the coupling device between the cap 14 and sealing stopper 15 described above, yet another coupling device may also be provided between the receiver container 2 and cap 14, although this is illustrated on a simplified basis only. It may comprise co-operating projections and indentations or alternatively threaded segments and/or thread webs, of the standard known type used for such receiver units.

The cap 14 has two end regions 25, 26 spaced apart from one another in the direction of the longitudinal axis 8, and in the embodiment illustrated as an example the open end region 25 extends around the open end face 13 of the receiver container 2 and the end face 13 runs close to or even abuts with the projection 17. In the position illustrated here, the end face 13 lies tightly against the surface of the projection 17 facing it.

Disposed on the internal surface 9 of the container wall 7 is at least one reinforcement rib 27 projecting in the direction towards the longitudinal axis 8. In this example of an embodiment, the at least one reinforcement rib 27 is disposed or arranged on at least a first subsection 28 of the internal surface 9 of the container wall 7, the first subsection 28 extending from the closed end 5 in the direction towards the open end 4. The at least one reinforcement rib 27 may also extend by its web end into the approximately spherically shaped base wall 6.

FIGS. 2 and 3 illustrate the receiver container 2 described above on its own and hence without the closure device 3, the same reference numbers and component names being used to describe parts that are the same as those described with reference to FIG. 1 above. To avoid unnecessary repetition, reference may be made to the more detailed description of FIG. 1 given above.

The receiver container 2 described and illustrated here has the first subsection 28 on its internal surface 9 extending from the closed end 5 in the direction towards the open end 4 on which the at least one reinforcement rib 27 is disposed or arranged. In this example of an embodiment, a second subsection 29 is also provided or arranged in the region of the open end 4 on the internal surface 9 of the container wall 7 adjoining the first subsection 28 and extending as far as the end face 13. The second subsection 29 is preferably of an approximately cylindrical design in at least certain regions relative to the longitudinal axis 8. The purpose of this is to provide the sealing surface described above in the form of a sealing section 30 on the internal surface 9 against which the sealing stopper 15 lies in a sealing arrangement by means of its sealing surface 21 and closes the receiving chamber 12.

The at least one reinforcement rib 27 has a spiral-shaped, in particular helical, longitudinal extension about the longitudinal axis 8 and as viewed in the direction of the longitudinal axis 8. However, it is preferable if several reinforcement ribs 27 are provided, uniformly distributed around the circumference. The number of reinforcement ribs 27 may be freely selected depending on requirements and, based on the standard dimensions currently in use, a number between 2 and 6 has proved to be of advantage. Furthermore, a pitch of the at least one reinforcement rib 27 based on its screw-shaped longitudinal extension may have a value selected from a range with a lower limit of 20 mm and an upper limit of 60 mm. The value of the pitch in this respect is based on a full circumference of 360°. The wall thickness 10 chosen for the load tests described below was 0.5 mm for standard dimensions of 13/75.

The smaller the selected pitch, the better the collapse behavior of the container wall when subjected to temperature over a longer period of time. For example, if the receiver container 2 is subjected to a temperature of less than 60° C. for a period of 20 hours [h], there is no ovalization of the cross-section to speak of. When subjected to a temperature of ca. 65° C. for a period of 20 hours [h], a slight degree of ovalization occurs but this does not lead to total collapse. The degree of ovalization may be e.g. between 15% and 20% starting from the circular cross-section. If the temperature is raised to 70° C., a total collapse will occur after only 270 seconds [s].

If several reinforcement ribs 27 are provided, it would also be possible for them to have different pitches from one another, which may mean that the reinforcement ribs 27 intersect one another, However, it would also be possible to select different pitch directions, e.g. right or left pitches. Irrespective of this, the mutual disposition and orientation of the reinforcement ribs 27 might be such that they intersect one another and form a sort of net at the intersection points.

As may be seen more clearly from FIG. 3, a width 31 of the at least one reinforcement rib 27 in the direction perpendicular to its longitudinal extension may have a value selected from a range of values with a lower limit of 0.5 mm, in particular 1.0 mm, and an upper limit of 4.0 mm. A protrusion 32 of the at least one reinforcement rib 27 above the internal surface 9 may have a value selected from a range of values with a lower limit of 0.05 mm and an upper limit of 0.6 mm. The value of the protrusion is preferably ca. 0.2 mm to 0.4 mm. As viewed in cross-section, the reinforcement rib 27 may have an arcuate cross-section. This cross-section could be formed by a segment of an arc, for example, and merge into the internal surface 9 at its edge.

In order to obtain a reduction in the wall thickness 10 of the container wall 7, the latter may have a value selected from a range of values with a lower limit of 0.3 mm and an upper limit of 0.8 mm. A preferred wall thickness 10 for the standard dimensions 13/75 may be 0.5 mm, for example. In the case of standard dimensions 13/100 and 16/100, the wall thickness 10 may be 0.7 mm, for example.

The base wall 6 may have a wall thickness that is the same as that of the container wall 7 or may also have a smaller or larger wall thickness. Due to the three-dimensional shape of the base wall 6, which is usually spherical or cambered, this section of the receiver container 2 already has a high intrinsic strength. The strength or stiffness of the container wall 7 with the reduced wall thickness 10 is increased by providing the at least one reinforcement rib 27. Accordingly, a saving can be made on the amount of material needed for the same external standard dimensions whilst at the same time imparting sufficient strength and stability in the region of the container wall 7.

The container wall 7 should be provided with a virtually continuously constant wall thickness 10, at least in its first subsection 28, with the exception of the at least one reinforcement rib 27 disposed or provided on it. As a result of this constantly thin wall thickness 10, an increase in the internal volume of the of the receiver container can be obtained whilst using receiver containers of the type used to date with the same three-dimensional shape and the same external dimensions. This also means that for the same suction volume as that of the standard receiver units used to date, a lesser vacuum or negative pressure has to be applied in order to obtain the same standard volume during filling. The reduction in vacuum also results in a lower flow speed when taking a blood sample and hence a more gentle operation. The risk of hemolysis is therefore also lower and the patient's vein is subject to less stress.

However, reducing the wall thickness 10 also leads to an increase in surface in the region of the receiving chamber 12. This also increases the reaction surface between the body fluid introduced into the receiving chamber 12, in particular blood, and the chemicals that are usually applied to the internal surface 9. Accordingly, the reactions of the blood introduced into the receiving chamber 12 and the chemical or chemicals already contained in it are accelerated, for example. The blood will therefore coagulate more rapidly, for example.

Providing the ribs or reinforcement ribs 27 also means that the surface directed towards the receiving chamber 12 is made larger. As explained above, receiver units, in particular blood specimen tubes, of several different sizes are currently available on the market and in use. The standard size or standard diameter is based on the external diameter of the receiver container, preferably in the region of its open end 4, and the length of the receiver container in the direction of its longitudinal axis 8. There are, naturally, slight variations in the actual diameters and lengths of the standard external dimensions specified below.

A first possible embodiment of the receiver container 2 has a standard diameter of 13 mm and a standard length of 75 mm. A second embodiment, for example, has a standard diameter of 13 mm and a standard length of 100 mm. However, there are also receiver containers with a standard diameter of 16 mm and a standard length of 100 mm.

Accordingly, a total length of the receiver container 2 in the direction of its longitudinal axis 8 may have a value selected from a range of values with a lower limit of 65 mm and an upper limit of 130 mm. Furthermore, an external diameter of the receiver container 2 in the region of its open end 4 may have a value selected from a range of values with a lower limit of 12 mm and an upper limit of 18 mm.

As may also be seen, the at least one reinforcement rib 27 is provided or disposed exclusively in the first subsection 28 of the internal surface 9 of the container wall 7.

Looking at and comparing individual ones of the existing standard receiver units currently in use, in the case of a standard size 13/75 which usually has a wall thickness of ca. 1.0 mm to 1.1 mm, the wall thickness 10 will now be reduced to a value of 0.8 mm and the surface area will be made larger by ca. 6%, for example. If the wall thickness 10 is even further reduced and is only 0.5 mm for example, and the reinforcement ribs 27 illustrated in FIG. 2 are provided in addition, this increase in surface compared with the known receiver containers based on the standard size of 13/75 will now be ca. 12%.

Increasing the size of the receiving chamber 12 by reducing the wall thickness 10 also means that if the standard receiver units used to date and the receiver units 1 with the slimmer wall thickness and having the at least one reinforcement rib 27 on the internal surface 9 are filled with the same quantities or volumes, the latter will have a lower liquid level when the two are placed in a vertical position.

Due to the fact that the holding capacity is now larger than that of the standard sizes used to date, it is now possible to use the standard size 13/75 of shorter length having the higher capacity instead of the standard size 13/100. Furthermore, in the case of some capacities, it would now be possible to switch from using the standard size 16/100 and use instead the standard size 13/100 having a smaller external diameter because the increase in capacity means that a larger quantity can be accommodated in the standard size 13/100. This is because the receiving chamber 12 and hence holding capacity of the newly proposed receiver container 2 enables a larger quantity to be introduced.

As a result, the maximum capacity of the standard size 13/100 with the reduced wall thickness 10 and the at least one reinforcement rib 27 is now 7 ml, for example.

Furthermore, a reduction in the space needed for transportation and storage purposes can now be obtained, which means that a larger quantity or number of sample materials can be stored in the same amount of space. A major factor, however, is the reduction in the cost of disposal for the end consumer, which is usually dependent on mass or weight.

Comparing the receiver units used to date based on the standard size 13/75 and the newly proposed receiver units 1 of the same standard size 13/75 but with a reduced wall thickness 10, the distance between the liquid level and the sealing stopper is bigger when filled with the same quantity, which means that the proportion by volume of this gap is also increased.

In accordance with standard ISO 6710 governing single-use blood specimen containers, for a standard capacity of ≧0.5 ml and less than 5 ml, a so-called headspace (corresponding to the volume of free space) of 25% of the total nominal contents (nominal volume) must be complied with. In the case of a filling quantity of ≧5 ml, the headspace must be 15% of the total nominal content. If the wall thickness 10 of the standard size 13/75 is reduced, for example to 0.8 mm, and without the reinforcement ribs 27, the headspace for the same filling quantity of 4 ml can already be increased by ca. 25% to 28%. If, on the other hand, the wall thickness 10 is reduced to a value of 0.5 mm and the number of reinforcement ribs 27 selected is four with a protrusion of 0.25 mm, the headspace is made larger by ca. 52% to 55% relative to the minimum headspace to be complied with. As a result of this increased headspace, however, a larger quantity can be accommodated before the headspace prescribed by the standard is reached.

As explained above, increasing the volume of the receiving chamber 12 also means that the degree of evacuation can be reduced compared with the standard sizes in use to date. For example, the standard size 13/75 used to date requires a degree of evacuation of ca. 80% to 84%, in particular 82%. In order to obtain the same suction volume, the degree of evacuation in the case of a wall thickness reduced to 0.8 mm can be reduced to a value of ca. 71% to 75%, in particular 73%. If the wall thickness is reduced to 0.5 mm, for example, and if four ribs are provided as described above, the degree of evacuation is reduced to ca. 64% to 68%, in particular 66%. Reducing the degree of evacuation also means that the energy used for evacuation purposes is also reduced. Due to the associated low pressure difference between the receiving chamber 12 and the external ambient pressure, however, any loss of the reduced pressure is delayed so that a longer storage period can be obtained.

Taking the internal pressure of the receiving chamber 12 in the case of a receiver unit 1 based on the standard size 13/75 and a standard filling quantity or standard volume of 4 ml relative to a base of 1000 mbar, the latter in the case of the standard tube used to date based on the standard size 13/75 will be between 180 mbar and 200 mbar, in particular 190 mbar, for example. Due to the reduction of the wall thickness to 0.8 mm and the associated increase in the volume of the receiving chamber 12, the internal pressure can be selected at a level that is higher by ca. 40% to 45%, in particular urn 42%, than the relatively low internal pressure of the original standard size 13/75. If the wall thickness 10 for the same standard size is further reduced to 0.5 mm and the four reinforcement ribs 27 described above are provided, the internal pressure can be selected so as to be even higher than the relatively low internal pressure of the original standard size 13/75 by ca. 76% to 82%, in particular 79%.

If one wanted to introduce a standard volume of 5 ml into a standard tube based on the standard size 13/75, this would not be possible due to the thicker wall thickness currently used. If the wall thickness were reduced to 0.8 mm, a headspace of ca. 17% can already be achieved with a filling volume of 5 ml. If, on the other hand, the wall thickness 10 is reduced to 0.5 mm and four of the helical reinforcement ribs 27 with a protrusion 32 of 0.25 mm are provided as described above, a headspace of 25% can be achieved.

FIG. 4 illustrates another and optionally independent embodiment of the receiver container 2 in its own right, the same reference numbers and component names being used to describe parts that are the same as those described with reference to FIGS. 1 to 3 above. To avoid unnecessary repetition, reference may be made to the more detailed description of FIGS. 1 to 3 given above.

In terms of its standard size, the receiver container 2 illustrated here corresponds to the standard diameter of 13 mm and a standard length of 100 mm. This receiver container 2 can be closed by means of the closure device 3 in the same was as described with reference to FIG. 1 to obtain the receiver unit 1.

The receiver container 2 has the open end 4 and the end 5 closed by the base wall 6. Extending between the two ends 4, 5 is the container wall 7, which defines the longitudinal axis 8. Again, several reinforcement ribs 27 are provided or disposed on the internal surface 9, which are of a spiral or helical shape and protrude out from the internal surface 9 in the direction towards the longitudinal axis 8.

Based on this example of an embodiment, the pitch of the individual reinforcement ribs 27 is selected as 30 mm. The selected number of reinforcement ribs 27 in this instance is four and they are distributed uniformly around the circumference.

Again in this instance, a first subsection 28 is provided, which extends from the closed end 5 along the container wall 7 in the direction towards the open end 4 but terminates short of the end face 13. Adjoining the first subsection 28 is the second subsection 29, which forms the sealing section 30 against which the sealing stopper 15 described above lies by means of its sealing surface 21.

To enable the wall thickness 10 to be reduced but impart dimensional stability and heat resistance, several reinforcement ribs 27 are provided, extending in a spiral or helical shape.

Irrespective of the above, it would also be possible for the pitch of the individual reinforcement ribs 27 to be based on a larger or smaller pitch than the one specified above.

FIG. 5 illustrates another and optionally independent embodiment of the receiver container 2 in its own right, the same reference numbers and component names being used to describe parts that are the same as those described with reference to FIGS. 1 to 4 above. To avoid unnecessary repetition, reference may be made to the more detailed description of FIGS. 1 to 4 given above.

The receiver container 2 illustrated here is again used to accommodate body fluids, in particular blood, and can be closed by means of the closure device 3, although this is not illustrated here.

Again, this receiver container 2 has the open end 4 and the end 5 closed by the base wall 6 and the container wall 7 defining the longitudinal axis 8 extends between the ends 4, 5. In this example of an embodiment, several reinforcement ribs 33 are provided on the internal surface 9 of the receiver container 2 disposed one after the other in the direction of the longitudinal axis 8 and spaced apart from one another. The various reinforcement ribs 33 are disposed in a plane oriented perpendicular to the longitudinal axis 8 and extend around the circumference, preferably continuously. The wall thickness 10 of the receiver container 2 between the internal surface 9 and the external surface 11 may be within the limits specified above.

As viewed in axial section, the reinforcement ribs 33 extend in a stepped pattern from the open end 4 towards the closed end 5.

Starting from the internal surface 9, a lightly sloping step-shaped transition stage 34 is provided which merges with an approximately cylindrically extending end face 35 of the at least one reinforcement rib 33. This end face 35 represents the largest protrusion of the reinforcement rib 33 above the internal surface 9. Adjoining the end face 35 is a transition section 36 which becomes wider from the open end 4 in the direction towards the closed end 5 and merges into or terminates at the internal surface 9. It should be noted that the expressions transition stage 34, end face 35 and transition section 36 relate to the disposition of the reinforcement rib 33 as viewed in axial section. Viewed in three dimensions, the end face 35 forms a cylindrical surface and the transition section 36 a forms a conical section, for example.

This cross-sectional shape of the reinforcement rib 33 described above could also be used for the reinforcement ribs 27 with the helical longitudinal extension described with reference to FIGS. 1 to 4, and this is indicated in FIG. 3 on the internal surface 9 in the bottom region by broken lines.

FIG. 6 illustrates another and optionally independent embodiment of the receiver container 2 in its own right, the same reference numbers and component names being used to denote parts that are the same as those described with reference to FIGS. 1 to 5 above. To avoid unnecessary repetition, reference may be made to the more detailed descriptions of FIGS. 1 to 5 given above.

The receiver container 2 described and illustrated here also has the open end 4 and the closed end 5, between which the longitudinal axis 8 extends. In the region of the first subsection 28 of the container wall 7, at least one of the reinforcement ribs 27 described with reference to FIGS. 1 to 5 is provided, projecting out from the internal surface 9. The wall thickness 10 in the first subsection 28 is reduced accordingly in the manner already described in connection with the different embodiments and standard sizes already described.

The second subsection 29 in this example of an embodiment is of a different design from that described above, although this design may be used with all of the embodiments described above.

The standard diameter described above (corresponding to the external nominal diameter) is denoted by reference 37 in this instance and may be either 13 mm or 16 mm. In order to impart sufficient strength or intrinsic stiffness to the open end 4 in the sealing section 30 and/or to prevent undesired penetration of the closed end 5 by another receiver container 2 due to its conical shape towards the closed end 5, at least the sealing section 30 additionally projects out beyond the internal surface 9 in the direction towards the longitudinal axis 8. The extra extension of the internal surface 9 is shown by broken lines in the left-hand part of the open end 5 in the container wall 7.

In this example of an embodiment, none of the reinforcement ribs 27 is provided in a first wall part 38 of the container wall 7 adjoining the first subsection 28 and the reduced, preferably constant wall thickness 10 in the direction towards the open end 4 extends as far as a transition section 39. The transition section 39 leading to the sealing section 30 may be provided in the form of transition radii and/or one or more frustoconical surfaces, for example, and because of the projection in the direction towards the longitudinal axis 8 in the region of the sealing section 30, another wall part 40 is formed which has a larger or thicker wall thickness 41 than the first wall part 38.

Due to the thicker design of the other wall part 40 and the resultant reduction of the clearance width, the open end 4 has an internal diameter 42 in this axial section that is smaller than an internal diameter in the first wall part 38 adjoining the transition section 39.

Irrespective of the above, it would also be possible for the first subsection 28 to extend into the region of the transition section 39 so that the at least one reinforcement rib 27 extends so as to directly adjoin the transition section 39. This possible embodiment is illustrated and will be described with reference to the next drawing, FIG. 7. The axial extension of the other wall part 40 is preferably selected so that it at least approximately corresponds to the axial extension of the sealing surface of the sealing stopper facing it.

Due to its projection, the other wall part 40 forms an undercut starting from the transition section 39 which has to be overcome when removing the mold core from the mold.

FIG. 7 illustrates the design and disposition of the reinforcement rib or ribs 27 outlined above. This may therefore be construed as another and optionally independent embodiment of the receiver container 2 in its own right, and again, the same reference numbers and component names are used to denote parts that are the same as those described with reference to FIGS. 1 to 6 above. To avoid unnecessary repetition, reference may be made to the more detailed descriptions of FIGS. 1 to 6 given above.

Since this embodiment is very similar to the one described in detail above with reference to FIG. 6, only the differences will be explained. Reference may be made to the description of FIG. 6 for aspects that remain unchanged. The design of the closed end 5 may be based on one of the designs described above and can be freely selected.

The container wall 7 in this instance likewise has the slimmer wall thickness 10 described above. Starting from the open end 4, the container wall 7 tapers in the region of its external surface 11 towards the closed end 5 where it terminates with a spherically shaped closed end 5. Again on the internal surface 9, several of the reinforcement ribs 27 are provided, extending in the direction of the longitudinal axis 8 in a helical pattern or arrangement.

Here too, the container wall 7 has the bigger wall thickness 41 in the region of the sealing section 30 and the internal diameter 42 is therefore reduced by this inwardly projecting wall section. The transition section 39 in this instance forms the transition between the slimmer wall thickness 10 and the wall thickness 41 larger than it and directly adjoins the sealing section 30 in the axial direction in the direction towards the closed end 5. The longitudinal extension of the transition section 39 is indicated by dimension arrow heads.

The first subsection 28 of the container wall 7 in this instance terminates in the region of the transition section 39 which lies closer to the open end face 13 of the receiver container 2. Accordingly, the reinforcement ribs 27 also extend close to or directly as far as the start of the sealing section 30.

FIG. 8 illustrates another and optionally independent embodiment of the receiver container 2 in the region of its closed end 5. The same reference numbers and component names are used to denote parts that are the same as those described with reference to FIGS. 1 to 7 above. To avoid unnecessary repetition, reference may be made to the more detailed description of FIGS. 1 to 7 given above.

As also illustrated here, the receiver container 2 has an external diameter 43 in the region of its closed end 5 in the transition region between the base wall 6 and the container wall 7. Due to the conicity of the receiver container 2 starting from the open end 4 with the standard diameter 37 towards the closed end 5 as described above, the external diameter 43 is smaller than the standard diameter 37.

As described with reference to FIGS. 6 and 7, the protrusion of the reinforced container wall in the region of the open end 4, namely in the region of the sealing section 30, is selected so that the internal diameter 42 in the region of the sealing section 30 is smaller than the external diameter 43 in the region of the closed end 5. This prevents the closed end 5 from penetrating the open end 4 of receiver containers 2 if they are disposed loosely next to one another so that receiver containers 2 cannot jam with one another or form a stack.

Based on this example of an embodiment, the base wall 6 has a base wall thickness 44 which more or less corresponds to the slimmer wall thickness 10 of the container wall 7. Due to the reduction in the wall thickness 10, in particular also the base wall thickness 44, the receiver container 2 or receiver unit 1 may be subjected to high stress during centrifuging, leading to undesired deformation of the base wall 6, which could be pushed inwards in the direction towards the receiving chamber 12.

To avoid or prevent such undesired deformation and thus prevent any media to be supplied and separated from undesirably getting out of the receiving chamber 12, webs 45 may be provided or disposed in the region of the internal surface 9 of the base wall 6, projecting out from the base wall 6 in the direction towards the receiving chamber 12. Several such webs 45 are preferably provided or disposed around the circumference on the base wall 6. The individual webs 45 may be disposed in various layouts relative to one another and in this instance a star-shaped arrangement has been selected extending out from a center of the base wall 6 formed by the longitudinal axis 8 to the circumferentially extending container wall.

FIG. 9 illustrates another possible and optionally independent embodiment of the receiver container 2 for forming the receiver unit 1, the same reference numbers and component names being used to denote parts that are the same as those described with reference to FIGS. 1 to 8 above. To avoid unnecessary repetition, reference may be made to the more detailed description of FIGS. 1 to 8 given above.

The container wall 7 has the same slim wall thickness 10 starting from the open end 4 having its end face 13 in the direction towards the closed end 5. This axial extension or longitudinal extension of the wall section having the slimmer wall thickness 10 has a value corresponding to approximately ⅔ of the standard length of the receiver container 2. This longitudinal section of the container wall 7 may also be described as the base longitudinal section 46 and is of a specific length. Disposed between the end of the base longitudinal section 46 facing the closed end 5 and the base wall 6 is a base-end longitudinal section 47 on the container wall 7. This base-end longitudinal section 47 of the container wall 7 has a wall thickness 48 which increases inwardly in the direction towards the closed end 5, as a result of which the internal dimension or internal diameter of the receiving chamber 12 in the base-end longitudinal section 47 becomes rapidly smaller than in the region of the base longitudinal section 46. The increase in the wall thickness 48 may be constant or linear.

As viewed in axial section, the external surface 11 of the container wall 7 is straight and also has a corresponding conicity relative to the longitudinal axis 8. In the region of the open end 4, the container wall 7 may have a slightly thicker or bigger wall thickness 10 in the region of its external surface 11 than in the region of the sealing section 30.

Due to the increase in the wall thickness 48 of the container wall 7 in the region of the base-end longitudinal section 47, the base wall 6 may also be provided with the corresponding base wall thickness 44. Accordingly, the base wall thickness 44 may entirely correspond to the wall thickness of receiver containers 2 known to date. For example, the base wall thickness 44 may have a value with a lower limit of 0.8 mm and an upper limit of 1.2 mm, preferably 1.0 mm.

The reinforcement ribs 27 described above may be provided or disposed on the internal surface 9 of the container wall 7. As viewed in the axial direction, the reinforcement rib(s) 27 terminate(s) just short of the sealing section 30, as illustrated and described with reference to FIG. 6 in connection with the second subsection 29. The first subsection 28 in which the reinforcement rib or ribs 27 is or are provided extends from the second subsection 29 in the direction towards the closed end 5 to approximately half the longitudinal extension of the base-end longitudinal section 47.

Due to the designs described above based on different wall thicknesses or wall thicknesses of the container wall 7 and/or base wall 6 in the direction of the longitudinal axis 8 and the provision of the reinforcement ribs 27 and/or webs 45 on the base wall 6 in at least certain regions, receiver containers 2 that are suitable for the intended purpose can be obtained. Consequently, it is possible to save on material resources by providing a slimmer wall thickness 10 at specific points or in specific longitudinal sections.

In order to obtain reduced wall thicknesses 10 for all the receiver containers 2 proposed by the invention in the manner described above, at least one barrier additive may be added to the plastic material, which is selected from the group comprising inorganic magnesium-aluminum silicates, ethylene-vinyl alcohol copolymers (EVOH), polyvinylidene chloride (PVDC), masterbatch or scavengers for improving the gas barrier properties. Irrespective of this or in addition, it would also be possible to apply a barrier coating to at least the internal surface 9 of the container wall 7, e.g. of SiOₓ. However, this barrier coating may also be applied to the external surface 11 or to both of the surfaces (internal surface 9 and external surface 11).

The embodiments illustrated as examples represent possible variants of the receiver unit 1, and it should be pointed out at this stage that the invention is not specifically limited to the variants specifically illustrated, and instead the individual variants may be used in different combinations with one another and these possible variations lie within the reach of the person skilled in this technical field given the disclosed technical teaching.

Furthermore, individual features or combinations of features from the different embodiments illustrated and described may be construed as independent inventive solutions or solutions proposed by the invention in their own right.

The objective underlying the independent inventive solutions may be found in the description.

All the figures relating to ranges of values in the description should be construed as meaning that they include any and all part-ranges, in which case, for example, the range of 1 to 10 should be understood as including all part-ranges starting from the lower limit of 1 to the upper limit of 10, i.e. all part-ranges starting with a lower limit of 1 or more and ending with an upper limit of 10 or less, e.g. 1 to 1.7, or 3.2 to 8.1 or 5.5 to 10.

Above all, the individual embodiments of the subject matter illustrated in FIGS. 1; 2, 3; 4; 5; 6; 7; 8; 9 constitute independent solutions proposed by the invention in their own right. The objectives and associated solutions proposed by the invention may be found in the detailed descriptions of these drawings.

For the sake of good order, finally, it should be pointed out that, in order to provide a clearer understanding of the structure of the receiver unit 1, it and its constituent parts are illustrated to a certain extent out of scale and/or on an enlarged scale and/or on a reduced scale.

List of reference numbers

| | |
|---|---|
| 1 | Receiver unit |
| 2 | Receiver container |
| | Closure device |
| 3 | Open end |
| 4 | Closed end |
| 5 | Base wall |
| 6 | Container wall |
| 7 | Longitudinal axis |
| 8 | Internal surface |
| 9 | Wall thickness |
| 10 | External surface |
| 11 | Receiving chamber |
| 12 | End face |
| 13 | Cap |
| 14 | Sealing stopper |
| 15 | Cap casing |
| 16 | Projection |
| 17 | Projection |
| 18 | Retaining ring |
| 19 | Shoulder |
| 20 | Sealing surface |
| 21 | Sealing surface |
| 22 | Recess |
| 23 | Opening |
| 24 | End region |
| 25 | End region |
| 26 | Reinforcement rib |
| 27 | First subsection |
| 28 | Second subsection |
| 29 | Sealing section |
| 30 | Width |
| 31 | Protrusion |
| 32 | Reinforcement rib |
| 33 | Transition stage |
| 34 | End face |
| 35 | Transition section |
| 36 | Standard diameter |
| 37 | Wall part |
| 38 | Transition section |
| 39 | Wall part |
| 40 | Wall thickness |
| 41 | Internal diameter |
| 42 | External diameter |
| 43 | Base wall thickness |
| 44 | Web |
| 45 | Base longitudinal section |
| 46 | Longitudinal section |
| 47 | Wall thickness |

1. Receiver container (2) for receiving body fluid, in particular blood, comprising an open end (4), an end (5) closed by a base wall (6), a container wall (7) which extends between the open end (4) and the closed end (5) and defines a longitudinal axis (8), the container wall (7) comprising an internal surface (9) and an external surface (11) spaced apart therefrom by a wall thickness (10), and the container wail (7) and the end (5) closed by the base wall (6) bound a receiving chamber (12), in which receiving chamber (12) the body fluid is directly received, and the receiver container (2) is made from a plastic material, wherein at least one reinforcement rib (27, 33) is provided on at least a first subsection (28) of the internal surface (9) of the container wall (7) and protrudes in the direction towards the longitudinal axis (8), and the first subsection (28) extends from the region of the closed end (5) in the direction towards the open end (4).

2. Receiver container (2) according to claim 1, wherein the at least one reinforcement rib (27) has a spiral-shaped, in particular helical, longitudinal extension about the longitudinal axis (8).

3. Receiver container (2) according to claim 2, wherein several reinforcement ribs (27) are provided, distributed uniformly around the circumference.

4. Receiver container (2) according to claim 2, wherein a pitch of the at least one reinforcement rib (27) relative to a full circumference of 360° has a value selected from a range of values with a lower limit of 20 mm and an upper limit of 60 mm.

5. Receiver container (2) according to claim 1, wherein several reinforcement ribs (33) are provided, disposed one after the other in the direction of the longitudinal axis (8) and spaced apart from one another, which reinforcement ribs (33) are disposed respectively in a plane perpendicular to the longitudinal axis (8) and extending around the circumference.

6. Receiver container (2) according to claim 1, wherein a width (31) of the at least one reinforcement rib (27, 33) in the direction perpendicular to its longitudinal extension has a value selected from a range of values with a lower limit of 0.50 mm and an upper limit of 4.00 mm.

7. Receiver container (2) according to claim 1, wherein a protrusion (32) of the at least one reinforcement rib (27, 33) above the internal surface (9) has a value selected from a range of values with a lower limit of 0.05 mm and an upper limit of 0.60 mm.

8. Receiver container (2) according to claim 1, wherein an external diameter of the receiver container (2) in the region of its open end (4) has a value selected from a range of values with a lower limit of 12 mm and an upper limit of 18 mm.

9. Receiver container (2) according to claim 1, wherein a total length of the receiver container (2) in the direction of its longitudinal axis (8) has a value selected from a range of values with a lower limit of 65 mm and an upper limit of 130 mm.

10. Receiver container (2) according to claim 1, wherein the wall thickness (10) of the container wall (7) has a value selected from a range of values with a lower limit of 0.3 mm and an upper limit of 0.8 mm.

11. Receiver container (2) according to claim 1, wherein the container wall (7), with the exception of the at least one reinforcement rib (27, 33) disposed thereon, has a virtually continuously constant wall thickness (10) at least in its first subsection (28).

12. Receiver container (2) according to claim 1, wherein the at least one reinforcement rib (27, 33) is disposed exclusively in the first subsection (28) of the internal surface (9) of the container wall (7).

13. Receiver container (2) according to claim 1, wherein a second subsection (29) of the internal surface (9) of the container wall (7) is disposed adjoining the first subsection (28), which second subsection (29) is designed as an approximately cylindrical sealing section (30) in at least certain regions.

14. Receiver container (2) according to claim 1, wherein at least the sealing section (30) projects out from the internal surface (9) of the container wall (7) in the direction towards the longitudinal axis (8), and a wall part (40) of the container wall (7) in the region of the sealing section (30) has a bigger wall thickness (41) than the wall thickness (10) of the first subsection (28) of the container wall (7).

15. Receiver container (2) according to claim 1, wherein an internal diameter (42) in the region of the wall part (40) constituting the sealing section (30) is smaller than an external diameter (43) of the container wall (7) in the region of the closed end (5).

16. Receiver container (2) according to claim 1, wherein at least one web (45) is provided on the base wall (6) on its internal surface facing the receiving chamber (12) and protruding from the internal surface thereof.

17. Receiver container (2) according to claim 1, wherein the base wall (6) has a bigger base wall thickness (44) than the wall thickness (10) of the container wall (7).

18. Receiver container (2) according to claim 1, wherein the container wall (7) at a base-end longitudinal section (47) has a wall thickness (48) that increases in the direction towards the base wall (6) relative to the rest of the wall thickness (10).

19. Receiver container (2) according to claim 1, wherein the plastic material is selected from the group comprising polyethylene terephthalate (PET), polyethylene (PE), polypropylene (PP), polystyrene (PS), polycarbonate (PC), high-density polyethylene (HD-PE), acrylonitrile-butadiene-styrene copolymers (ABS), polyamides (PA).

20. Receiver container (2) according to claim 1, wherein at least one barrier additive is added to the plastic material, e.g. inorganic magnesium-aluminum silicates, ethylene-vinyl alcohol copolymers (EVOH), polyvinylidene chloride (PVDC), masterbatch or scavengers, to improve the gas barrier properties.

21. Receiver container (2) according to claim 1, wherein a barrier coating, in particular SiOₓ, is provided on or applied to at least the internal surface (9) and/or the external surface (11) of the container wall (7).

22. Receiver unit (1) for receiving body fluid, in particular blood, comprising a receiver container (2) with an open end (4), a closed end (5) and a container wall (7) extending between the open end (4) and the closed end (5), and a closure device (3) by means of which the open end (4) of the receiver container (2) is closed, wherein the receiver container (2) is as specified in claim 1.

23. Receiver unit (1) according to claim 18, wherein a receiving chamber (12) sealed off from the external ambient atmosphere is reduced to a pressure below atmospheric pressure.
